SEQUENCE LISTING (13 sequences)

SEQ ID NO: 1 - protein, 9 aa, synthetic construct
QGMAPAAGA

SEQ ID NO: 2 - protein, 10 aa, synthetic construct
LQGMAPAAGA

SEQ ID NO: 3 - protein, 10 aa, synthetic construct
GMAPAAGADL

SEQ ID NO: 4 - protein, 10 aa, synthetic construct
LQGLAPLGSE

SEQ ID NO: 5 - protein, 10 aa, synthetic construct
LQGMAYTVQG

SEQ ID NO: 6 - protein, 10 aa, synthetic construct
  MOD_RES 10: note = biotinylated with optional linker
QGMAPAAGAL

SEQ ID NO: 7 - protein, 11 aa, synthetic construct
RLQGMAPAAGA

SEQ ID NO: 8 - protein, 30 aa, synthetic construct
DSMDRIEKDRLQGMAPAAGADLDKIGLHSD

SEQ ID NO: 9 - protein, 11 aa, synthetic construct
GMPAPAAGADL

SEQ ID NO: 10 - protein, 13 aa, synthetic construct
  MOD_RES 13: note = linked to Keyhole Limpet Hemocyanin
LQGMAPAAGAGGC

SEQ ID NO: 11 - protein, 12 aa, synthetic construct
  MOD_RES 12: note = linked to Keyhole Limpet Hemocyanin
DCTSTFPRVGGC

SEQ ID NO: 12 - protein, 30 aa, synthetic construct
QGMAPAAGADLDKIGLHSDSQEELWMFVRK

SEQ ID NO: 13 - protein, 77 aa, Homo sapiens
EEVRKLKARVDELERIRRSILPYGDSMDRIEKDRLQGMAPAAGADLDKIGLHSDSQEELWMFVRKKLMMEQENGNLR

(19) United States
(12) Patent Application Publication
(10) Pub. No.: US 2026/0266825 A1
(43) Pub. Date: Sep. 10, 2026

(54) PRO-C17 Assay

(71) Applicant: Nordic Bioscience A/S, Herlev (DK)

(72) Inventors: Nicholas Willumsen, Gentofte (DK); Marina Crespo Bravo, Herlev (DK); Rasmus Sund Petersen, Herlev (DK); Morten Asser Karsdal, Kobenhavn Ø (DK)

(73) Assignee: Nordic Bioscience A/S, Herlev (DK)

(21) Appl. No.: 19/167,380

(22) PCT Filed: Mar. 22, 2024
(86) PCT No.: PCT/EP2024/057775

§ 371 Date: Sep. 21, 2025

(30) Foreign Application Priority Data
Mar. 22, 2023 (GB) 2304208.8

(51) Int. Cl. G01N 33/57535

(52) U.S. Cl. CPC G01N 33/57535; G01N 2470/04; G01N 2470/10

(57) ABSTRACT

Described herein is a method of immunoassay (the “PRO-C17 assay”) for measuring the levels of type XVII collagen ectodomain present in a patient sample, which method of immunoassay can be used for detecting and/or monitoring cancer and/or assessing the severity thereof in a patient. Also described are monoclonal antibodies and assay kits suitable for use in carrying out said methods.

### FIELD OF INVENTION

The present invention relates to methods of immunoassay that are suitable for use in detecting and/or monitoring cancer and/or assessing the severity thereof, and well as antibodies and assay kits that are suitable for use in carrying out said methods.

### BACKGROUND

Cancer is the world's leading cause of death, accounting for almost 10 million deaths in 2020. The use of biomarkers is critical for early diagnosis as well as selection of appropriate treatments to reduce the burden of cancer^{[1]}. In recent years, attention has been drawn towards the tumour microenvironment and more specifically to the extracellular matrix (ECM), which has been reported to facilitate tumour progression and reduce the response to cancer treatments^{[2]}.

Collagens are the main component of the ECM and to date 28 different collagens have been identified^{[3]}. In healthy tissue, collagen production and degradation is highly regulated to maintain tissue homeostasis. In cancer, however, excessive turnover and remodelling occurs, resulting in loss of tissue organizational cellular behaviour leading to disease progression and imbalanced angiogenesis that can promote tumorigenesis^{[4]}. Protein fragments resulting from this excessive ECM remodelling are released into circulation and can be measured non-invasively in a liquid biopsy and be used as biomarkers. Even though extensive research and biomarker discovery has been directed towards the most predominant collagens of the ECM such as type I, III, and IV, little is known about less abundant highly specialized group of collagens.

Membrane-associated collagens with interrupted triple-helices (MACITs), including collagen types XIII, XVII, XXIII and XXV, are type II proteins that contain large ectodomains that are not present in other collagens and that can be shed and released into the pericellular matrix^{[5,6]}. Overall, MACITs are expressed in low quantities in adult tissue but are upregulated during both embryogenesis and tumorigenesis. Even though MACITs can contribute to maintaining ECM stability, growing evidence demonstrates that they play essential roles in cell fate processes by binding to ECM components such as cell surface receptors or growth factors, both in healthy and cancerous tissue^{[7-9}. MACITs consists of an N-terminal cytoplasmatic domain, a transmembrane domain and a C-terminal ectodomain consisting of different collagenous domains (COL) and non-collagenous domains (NC)^{[10]}. Collagen XIII, XXIII and XXV are similar in structure and their ectodomain is cleaved by furin proteases, whereas collagen XVII differs structurally and is cleaved by ADAM 9, 10 and 17^{[11-13]}.

Type XVII collagen, also known as BP180 or BPAG2, consists of three identical 180-kDa α-chains and is part of the hemidesmosomes, providing stable adhesion between the basal keratinocytes and the basement membrane and thus playing a crucial role in the skin^{[14]}. Several blistering skin diseases have been associated with both genetic and acquired dysfunctions of type XVII collagen. For example, mutations in the COL17A1 gene can cause junctional epidermolysis bullosa (JEB) whereas autoimmunity towards the NC16A domain of shed type XVII collagen induces bullous pemphigoid (BP)^{[15-17]}. In one study, it was shown that polyclonal antibodies raised against the peptide sequence LQGMAPAAG (corresponding to aa 524-532 in the NC16A domain of type XVII collagen) preferentially react with the shed ectodomain of type XVII collagen, and that the sera of bullous pemphigoid react with this same peptide, suggesting that a neoepitope of the shed ectodomain of type XVII collagen is located in this stretch of amino acids and may serve as a target for blister inducing antibodies^{[24]}.

In cancer, type XVII collagen and its shed ectodomain appear to play a major role in epithelial cancer development and metastasis^{[18]}. Abnormal expression of the COL17A1 gene has been detected in many epithelial tumours such as colorectal, pancreatic, breast, ovarian or squamous cell carcinoma and associated with poor prognosis^{[19-22]}. Furthermore, a recent study into the role of type XVII collagen in cancer stem cell dormancy revealed that COL17A1-KO colorectal cancer organoids tended to form bigger colonies than the wild-type ones because of an increased proliferation rate. They also detected that COL17A1-KO colorectal cancer organoids lost dormant LGR⁺p27⁺ cells and were more sensitive to chemotherapy suggesting that COL17A1 could play a role in the maintenance of cell dormancy^{[23]}.

### SUMMARY OF THE INVENTION

The inventors have developed and validated an ELISA targeting the type XVII collagen ectodomain (also referred to herein as the “PRO-C17 assay”), and have measured the levels of PRO-C17 (i.e. the levels of the type XVII collagen ectodomain as detected by the PRO-C17 assay) in circulation in serum samples to study the potential of PRO-C17 as biomarker for patients with cancer. The assay was found to be technically robust, and the data showed that PRO-C17 levels were significantly elevated in all cancer types and that colorectal cancer patients with high PRO-C17 levels had a lower overall survival and increased risk of dying.

Accordingly, in a first aspect the present invention provides a method of immunoassay, said method comprising:
i) contacting a patient sample with a monoclonal antibody that specifically binds to the amino acid sequence QGMAPAAGA;
ii) detecting and determining the amount of binding between the monoclonal antibody and peptides in the sample.

In a preferred embodiment, the method is a method of immunoassay for detecting and/or monitoring a cancer in a patient and/or assessing the severity of a cancer in a patient, the method further comprising;
iii) correlating the amount of binding with values associated with normal healthy subjects and/or values associated with known disease severity and/or values obtained from the patient at a previous time point and/or with a predetermined cut-off value.

In a preferred embodiment, the monoclonal antibody specifically binds to the amino acid sequence LQGMAPAAGA.

In a preferred embodiment, the monoclonal antibody does not specifically bind to the amino acid sequence GMAPAAGADL (i.e. a truncated version of the QGMAPAAGA sequence that has been truncated by removal of the N terminus glutamine residue). Preferably, the ratio of the affinity of said antibody for the QGMAPAAGA sequence to the affinity of said antibody for the GMAPAAGADL truncated sequence is at least 10 to 1, and more preferably is at least 20 to 1, at least 30 to 1, at least 40 to 1, at least 50 to 1 or at least 100 to 1.

In a preferred embodiment, the monoclonal antibody does not specifically bind to the amino acid sequence LQGLAPLGSE. Preferably, the ratio of the affinity of said antibody for the QGMAPAAGA sequence to the affinity of said antibody for the LQGLAPLGSE sequence is at least 10 to 1, and more preferably is at least 20 to 1, at least 30 to 1, at least 40 to 1, at least 50 to 1 or at least 100 to 1.

In a preferred embodiment, the monoclonal antibody does not specifically bind to the amino acid sequence LQGMAYTVQG. Preferably, the ratio of the affinity of said antibody for the QGMAPAAGA sequence to the affinity of said antibody for the LQGMAYTVQG sequence is at least 10 to 1, and more preferably is at least 20 to 1, at least 30 to 1, at least 40 to 1, at least 50 to 1 or at least 100 to 1.

In a preferred embodiment, the monoclonal antibody is raised against a synthetic peptide comprising the amino acid sequence LQGMAPAAGA. Preferably, the synthetic peptide consists of the amino acid sequence LQGMAPAAGA. For example, the monoclonal antibodies may be raised by: (a) immunizing a rodent (or other suitable mammal) with a synthetic peptide comprising the amino acid sequence LQGMAPAAGA, which peptide may optionally be linked at its C terminus to an immunogenic carrier protein (such as keyhole limpet hemocyanin); (b) isolating and cloning a single antibody producing cell; and (c) assaying the resulting monoclonal antibodies to ensure that they have the desired specificity.

In a preferred embodiment, the cancer is pancreatic cancer, colorectal cancer, kidney cancer, ovarian cancer, bladder cancer, lung cancer, melanoma, breast cancer, head and neck cancer, prostate cancer, or stomach cancer. Preferably, the cancer is colorectal cancer, kidney cancer, ovarian cancer, bladder cancer, breast cancer, or head and neck cancer. Most preferably, the cancer is colorectal cancer.

As noted above, in some embodiments the method is a method of immunoassay for assessing the severity of a cancer in a patient. For example, the method may be method for detecting a level of severity associated with an average overall survival time.

In a preferred embodiment, the patient sample is a human biofluid sample. Preferably the sample is a blood-based sample, such as blood (whole blood), plasma or serum.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

As used herein the term “N-terminus” refers to an N-terminal peptide sequence at the extremity of a polypeptide, i.e. at the N-terminal end of the polypeptide, and is not to be construed as meaning in the general direction thereof. As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGl, IgG2, IgG3, and IgG4 in the case of humans and IgGl, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The CDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. a cancer) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without cancer); and the term “values associated with known disease severity” means standardised quantities of binding determined by the method described supra for samples from patients known to have disease (i.e. a cancer) of a known severity.

In a second aspect, the present invention provides a method of treating a cancer in a patient in need thereof, the method comprising;
(a) carrying out a method of immunoassay in accordance with the first aspect of the present invention on a sample from a patient; and
(b) administering to the patient a therapy for the treatment of a cancer if it is determined in step
(a) that the patient has a cancer or a particular level of severity thereof.

Preferred embodiments of the method in accordance with the second aspect will be apparent from the foregoing discussion of preferred embodiments of the methods according to the first aspect. For example, step (a) may in particular comprise carrying out the method of immunoassay to detect a bladder cancer, breast cancer, colorectal cancer, head and neck cancer, kidney cancer, lung cancer, melanoma, ovarian cancer, pancreatic cancer, prostate cancer or stomach cancer, or a particular level of severity thereof.

The therapy may be any therapy suitable for treating the cancer in question. The therapy may for example comprise or consist of one or more surgeries, one or more radiation therapies, one or more medicaments (such as for example one or more chemotherapies, one or more immunotherapies and/or one or more hormonal therapies), or combinations thereof. Medicaments may be formulated for topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, debulking surgeries, palliative surgeries and/or restorative surgeries.

For example, where the cancer is bladder cancer, suitable therapies may comprise one or more of: transurethral resection of bladder tumour (TURBT) with or without intravesical chemotherapy or immunotherapy; radical cystectomy plus neoadjuvant chemotherapy or transurethral resection with chemoradiation or partial cystectomy plus neoadjuvant chemotherapy; cisplatin-based chemotherapy, optionally followed by radical cystectomy or chemoradiation; carboplatin-based chemotherapy; immune checkpoint inhibitors; radical cystectomy; and palliative radiotherapy.

Where the cancer is breast cancer, suitable therapies may for example comprise one or more of: a mastectomy, quadrantectomy or lumpectomy; estrogen receptor blockers (such as tamoxifen); aromatase inhibitors (such as anastrozole or letrozole) that block production of estrogen; CDK inhibitors; one or more chemotherapeutic agents such as a combination of cyclophosphamide, doxorubicin and optionally a taxane (such as docetaxel), or a combination of cyclophosphamide, methotrexate, and fluorouracil; one or more monoclonal antibodies such as trastuzumab and/or pertuzumab; and radiotherapy.

Where the cancer is colorectal cancer, suitable therapies may for example comprise one or more of: endoscopic mucosal resection or endoscopic submucosal dissection; a partial colectomy (or proctocolectomy for rectal lesions); chemotherapy agents such as for example capecitabine, fluorouracil, irinotecan, oxaliplatin or UFT; antiangiogenic drugs such as for example bevacizumab; epidermal growth factor receptor inhibitors, such as for example aflibercept, cetuximab and panitumumab; radiation therapy; immune checkpoint inhibitors; and monoclonal antibodies such as pembrolizumab or dostarlimab.

Where the cancer is head and neck cancer, suitable therapies may for example comprise one or more of: surgery, including but not limited to laser surgery; radiation therapy, including but not limited to 3D conformal radiation therapy, intensity-modulated radiation therapy, particle beam therapy and brachytherapy; one or more chemotherapy agents such as for example, paclitaxel, carboplatin, cetuximab, docetaxel, cisplatin and fluorouracil; photodynamic therapy utilizing amphinex; monoclonal antibodies such as cetuximab, bevacizumab, erlotinib, pembrolizumab or nivolumab; gene therapies such as gendicine; and immune checkpoint inhibitors. Where the cancer is kidney cancer, suitable therapies may for example comprise one or more of: whole kidney removal or partial removal of the kidney, by surgery; freezing the tumour or treating it with high temperatures; biological therapies such as everolimus, torisel, nexavar, sutent, axitinib, sunitinib, pazopanib, sorafenib, cabozantinib and/or lenvatinib; immunotherapy using interferon and/or interleukin-2; monoclonal antibodies such as nivolumab; immune checkpoint inhibitors; and radiotherapy.

Where the cancer is lung cancer, suitable therapies may for example comprise one or more of: surgery, such as performing a lobectomy, a sublobar excision (wedge resection) or removal of a whole lung (pneumonectomy); radiotherapy, examples of which include radiotherapy given together with chemotherapy, post-operative radiotherapy, brachytherapy (localized radiotherapy) given directly inside the airway, prophylactic cranial irradiation, stereotactic radiation and palliative radiotherapy; chemotherapy using for example one more agents such as cisplatin/carboplatin, etoposide, gemcitabine, paclitaxel, docetaxel, vinorelbine, topotecan, irinotecan and pemetrexed; epidermal growth factor receptor (EGFR) inhibitors drugs such as erlotinib, gefitinib, afatinib, dacomitinib or osimertinib; and immunotherapy, using for example anti PD-L1 monoclonal antibodies such as atezolizumab, nivolumab or pembrolizumab, monoclonal antibodies targeting cytotoxic T-lymphocyte-associated protein 4 (CTLA-4) such as ipilimumab, and/or monoclonal antibodies that targets vascular endothelial growth factor such as bevacizumab.

Where the cancer is melanoma, suitable therapies may for example comprise one or more of: surgical excision of the tumour, and optionally lymph nodes in the area of the tumour; interferon treatment; chemotherapy using agents such as for example dacarbazine; small-molecule targeted therapies using for example BRAF inhibitors (such as vemurafenib and dabrafenib), MEK inhibitors (trametinib), C-Kit inhibitors and/or NRAS inhibitors; immunotherapy using cytokines (e.g. IL-2 and/or IFN-α), immune check point inhibitors (such as for example anti-CTLA-4 monoclonal antibodies such as ipilimumab or tremelimumab, toll-like receptor (TLR) agonists, CD40 agonists, anti-PD-1 antibodies such as pembrolizumab, pidilizumab or nivolumab, LAG-3 inhibitors such as relatlimab, and/or PD-L1 antibodies), and/or adoptive cell transfer (using for example pre-stimulated, modified T cells or dendritic cells); and radiotherapy.

Where the cancer is ovarian cancer, suitable therapies may for example comprise one or more of: removal of one (unilateral oophorectomy) or both ovaries (bilateral oophorectomy), and optionally also the fallopian tubes (salpingectomy), uterus (hysterectomy) and/or the omentum (omentectomy); debulking surgery; chemotherapy (including neoadjuvant or adjuvant chemotherapy) using agents such as paclitaxel, cisplatin, topotecan, doxorubicin, epirubicin, gemcitabine, carboplatin, docetaxel, vincristine, dactinomycin, etoposide, cyclophosphamide, oxaliplatin or combinations thereof; radiotherapy; hormonal therapy; and immunotherapy, such as for example the antibody drug bevacizumab.

For example where the cancer is a pancreatic cancer, such as for example PDAC, suitable therapies may for example comprise one or more of: surgical resection, such as for example tumor resection, the Whipple procedure, total pancreatectomy or distal pancreatectomy; radiotherapy, examples of which include but are not limited to radiotherapy given together with chemotherapy, post-operative radiotherapy, brachytherapy (localized radiotherapy), stereotactic radiation and palliative radiotherapy; chemotherapy using for example one more agents such as gemcitabine, 5-FU, erlotinib, FOLFIRINOX, nab-paclitaxel or combinations thereof; the somatostatin analog class of medications; lanreotide; targeted therapy using everolimus or sunitinib; nuclear medicine therapy with radiolabeled peptides or hormones such as iobenguane; and techniques such as radiofrequency ablation (RFA), cryoablation, or hepatic artery embolization.

Where the cancer is prostate cancer, suitable therapies may for example comprise one or more of: radiotherapy; chemotherapy using chemotherapeutic agents, such as for example docetaxel, cabazitaxel, docetaxel, thalidomide, and combinations thereof; immunotherapy, such as for example the monoclonal antibody bevacizumab; hormonal therapies such as for example abiraterone and enzalutamide; external beam radiation therapy; particle therapy; high-intensity focused ultrasound; cryotherapy; and surgical procedures such as for example a radical prostatectomy.

Where the cancer is stomach cancer, suitable therapies may for example comprise one or more of: surgical procedures such as for example an endoscopic mucosal resection. endoscopic submucosal dissection or gastrectomy; chemotherapy using agents such as fluorouracil, capecitabine, BCNU, methyl-CCNU, and doxorubicin, mitomycin C, cisplatin, Taxotere or combinations thereof; targeted therapy using epidermal growth factor receptor 2 inhibitors, such as trastuzumab; and radiotherapy.

In a third aspect, the present invention provides a monoclonal antibody that specifically binds to the amino acid sequence QGMAPAAGA. The antibody according to the third aspect of the invention is, in particular, suitable for use in carrying out the methods of immunoassay according to the first aspect of the invention. Preferred embodiments and features of the antibody according to the third aspect will therefore be apparent from the above discussion of the preferred embodiments of the methods according to the first aspect.

In a fourth aspect, the present invention provides an immunoassay kit comprising a monoclonal antibody in accordance with the third aspect of the present invention, and at least one of:
a streptavidin coated well plate
a biotinylated peptide QGMAPAAGA-L-Biotin wherein L is an optional linker
a secondary antibody for use in a sandwich immunoassay
a calibrator protein comprising the amino acid sequence QGMAPAAGA
an antibody biotinylation kit
an antibody HRP labelling kit
an antibody radiolabelling kit

The immunoassay kit according to the fourth aspect of the invention is, in particular, suitable for use in carrying out the method of immunoassay according to the first aspect of the invention. Further preferred embodiments and features of the immunoassay kit according to the fourth aspect will therefore be apparent from the above discussion of the preferred embodiments of the methods according to the first aspect.

### FIGURES

FIG. 1: Specificity of the PRO-C17 monoclonal antibody. The reactivity of the monoclonal antibody used in the competitive PRO-C17 ELISA was evaluated toward the selection peptide (LQGMAPAAGA) and two deselection peptides (LQGMAYTVOG and LQGLAPLGSE). The monoclonal antibody was also specific for an elongated peptide (RLQGCMAPAAGA) and a truncated peptide (QGMAPPAGA). B/B0 is the ratio between the OD when the analyte is present (B) and the maximum OD when the analyte is not present (B0).

FIG. 2: Depiction of the NC16A domain of type XVII collagen. Physiological cleavage sites are indicated with black arrows and selection peptide is highlighted.

FIG. 3: Specificity of the PRO-C17 monoclonal antibody toward a further elongated peptide DSMDRIEKDRLQGMAPAAGADLDKIGLHSD) and a further peptide (GMPAPAAGADL) selected from the epitope mapping. The antibody was specific for the further elongated peptide but not for the further truncated peptide.

FIG. 4: Western blot results of type XVII collagen in supernatant and cell lysate from A-431 cells with the commercial type XVII antibody (right) and the PRO-C17 antibody (left). In supernatant only the 120 kDa fragment corresponding to the type XVII ectodomain was detected. The 180 kDa fragment corresponding to the full size of type XVII collagen was detected in the cell lysate.

FIG. 5: Type XVII collagen measured in non-small cell lung cancer (NSCLC) patients compared to healthy controls with the PRO-C17 assay (left) and with the commercial type XVII collagen kit (right) The comparison was performed with a parametric t-test. (ns=not significant).

FIG. 6: Correlation between type XVII collagen levels using the PRO-C17 assay and the type XVII commercial assay. Pearson's correlation coefficient (r) was −0.007357 (p>0.05, 95% CI −0.5012 to 0.4902)

FIG. 7: PRO-C17 levels in serum of cancer patients. Quantification of PRO-C17 in the serum of healthy controls (n=23) and bladder cancer (n=19), breast cancer (n=19), colorectal cancer (n=20), head & neck cancer (n=20), kidney cancer (n=20), lung cancer (n=17), melanoma (n=20), ovarian cancer (n=20), pancreatic cancer (n=20), prostate cancer (n=19) and stomach cancer (n=20). PRO-C17 levels are presented as Tukey-style boxplots with data-point jitter: horizontal bars indicate the median; upper- and lower hinges of the box indicate the first and third quartiles (the 25th and 75th percentiles); whiskers extend from the hinges to the largest or smallest value but no further than 1.5*IQR (where IQR is the inter-quartile range between the first and third quartiles) in either the positive or negative direction. Samples measuring below the LLOQ were given the value of LLOQ, as determined in the validation of PRO-C17. Differences in PRO-C17 levels between the cancer-groups and the healthy controls were evaluated by ordinary ANOVA followed by Dunnett's multiple comparisons t-test. *** indicates a p-value below 0.0001. *** below 0.001. * * below 0.01. * below 0.05.

FIG. 8: PRO-C17 levels measured in colorectal cancer patients. PRO-C17 levels were significantly elevated in patients with metastatic colorectal cancer (n=212) compared to healthy controls (n=23). The comparison was performed with a parametric t-test. **** indicates p<0.0001.

FIG. 9: Kaplan-Meier survival plots. High levels of PRO-C17 were associated with shorter overall survival (OS) in patients with metastatic colorectal patients. Upper line shows low PRO-C17 levels (tertile 1+tertile 2) and lower line shows high PRO-C17 levels (tertile 3).

FIG. 10: Hazard ratios (HR). HR were calculated by multivariate Cox proportional-hazards analysis stratified by line of treatment.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Antibody Development for PRO-C17

A 10 amino-acid target peptide ⁵²⁴LQGMAPAAGA⁵³³, corresponding to aa 524-533 in the NC16A domain of type XVII collagen (UniprotKB: Q9UMD9), was purchased from Genscript (Piscataway, NJ, USA) and used for immunization.

More specifically, an immunogenic peptide (LQGMAPAAGA-GGC-“KLH”) was generated by covalently cross-linking the target peptide to Keyhole Limpet Hemocyanin (“KLH”) carrier protein using sulfosuccinimidyl 4-(N-maleimidomethyl)cyclohexane-1-carboxylate, SMCC (Thermo Scientific, Waltham, MA, USA, cat.no. 22322). Glycine and cysteine residues were added at the C-terminal end of the target peptide as a linker to ensure correct linking of the carrier protein to the target peptide.

Monoclonal antibodies were generated by subcutaneous immunization of six-week-old Balb/C mice with 200 μL emulsified antigen containing 100 μg immunogenic peptide mixed with Sigma Adjuvant System (Sigma cat. No. S6322) Specol (Invitrogen cat.no. 7925000). Consecutive immunizations were performed at 2-week intervals until stable sera titer levels were reached. The mouse with the highest titer was chosen for fusion and rested for four weeks before being boosted with 100 ug immunogenic peptide in 100 μL 0.9% NaCl solution intravenously. Hybridoma cells were then produced by fusing spleen cells with SP2/0 myeloma cells as previously described (Gefter, Margulies and Scharff, 1977). The resultant hybridoma cells were then cultured in 96-well microtiter plates and standard limited dilution was used to secure monoclonal growth.

The monoclonal antibodies were purified using protein-G-columns according to the manufacturer's instructions (GE Healthcare Life Sciences, Little Chalfont, UK, cat. #17-0404-01).

The best antibody clone for the biomarker was selected based on a preliminary competitive ELISA for the reactivity towards the target peptide (LQGMAPAAGA), an elongated peptide (RLQGMAPAAGA), a truncated peptide (QGMAPAAGA), two deselection peptides (LQGMAYTVQG and LQGLAPLGSE), a target KLH-conjugated peptide (LQGMAPAAGA-GGC-KLH) and a non-sense KLH-conjugated peptide (DCTSTFPRV-GGC-KLH). Supernatant from the chosen clone was harvested and the monoclonal antibody (mAb) was purified and used for assay development.

### PRO-C17 ELISA Protocol

The development of a competitive ELISA (referred to herein as the “PRO-C17 assay”) employing the mAb described above included several preliminary optimizing experiments where several assay buffers, incubation time and temperature as well as concentrations of antibodies and peptides were tested.

The final PRO-C17 protocol was as follows: a 96-well streptavidin-coated ELISA plate was coated with 100 μL/well of 2.5 ng/ml of biotinylated QGMAPAAGA peptide dissolved in assay buffer (50 mM PBS, 1% BSA (w/v), 0.018% bronidox (v/v), 0.1% Tween-20 (w/v), 8 g/L NaCl, pH 7.4) incubated with shaking (300 rpm) for 30 minutes at 20° C. in darkness. After washing 5 times with washing buffer (25 mM Tris, 50 mM NaCl, pH 7.2), 20 μL/well of pre-diluted sample (1:2) was added in duplicates followed by 100 μL/well of 25 ng/mL HRP-labelled monoclonal antibody in assay buffer and incubated with shaking (300 rpm) for 20 hours at 4° C. in darkness. After a second washing cycle, 100 μL/well of TMB was added and incubated with shaking (300 rpm) for 15 minutes at 20° C. in darkness. The reaction was stopped by adding 100 μL/well of 1% H₂SO₄. Absorbance was measured at 450 nm with 650 nm as reference. The standard curve was generated by adding 20 μl/well of 30 ng/ml QGMAPAAGADLDKIGLHSDSQEELWMFVRK peptide serially diluted twofold and a four-parameter logistic (4PL) model was used to fit a curve. Each plate included 5 quality control samples that included pig serum (1), human serum (2), donkey serum (1) and peptide in assay buffer (1) to monitor inter- and intra-assay variation.

### Technical Evaluation of the PRO-C17 ELISA

The specificity of the antibody and assay towards the target peptide (LQGMAPAAGA) was assessed by including elongated (RLQGMAPAAGA) and truncated (QGMAPAAGA) versions of the peptide as well as two deselection peptides (LQGMAYTVQG and LQGLAPLGSE). Linearity was validated by performing serial dilutions of serum samples in assay buffer and calculating the percentage recovery relative to the dilution. Accuracy of the assay was evaluated by spiking a serum sample with high concentration of the analyte with another serum sample with low concentration at different ratios (100:0, 75:25, 50:50, 25:75, 0:100) and calculating the percentage recovery relative to the analyte concentrations when the samples were measured separately. Interference towards the most common endogenous analytes (hemoglobin, biotin and lipids) was evaluated by spiking serum samples with known amounts of the interfering substances (hemoglobin low=2.5 mg/mL, high=5 mg/mL; lipids low=1.5 mg/mL, high=5 mg/ml; biotin low=5 ng/ml, high=100 ng/ml) and calculating the percentage of recovery compared to the non-spiked sample. The variation was determined by running 10 independent plates including 3 human serum samples, 3 quality control samples (QCs), 2 peptides in assay buffer and 2 kit controls (COs) in double determinations. Intra-assay variation was calculated as the mean CV % between the sample replicates within the same plate. Intra-assay variation was accepted at CV %<10. Inter-assay variation was calculated as the mean CV % between runs of samples replicates on different plates and the acceptance criteria was CV %<158. Lower and upper limit of measurement range (LLOQ and ULOQ) were defined as the maximum and minimum concentrations measured with precision in the linear range of the assay. Analyte stability was assessed by storing three serum samples for 2, 4, 24, and 48 h at both 4 and 20° C. and calculating the percentage of recovery compared to the corresponding control samples stored at −20° C. Freeze-thaw stability determined by freezing and thawing serum samples multiple times (4 cycles) and calculating the percentage of recovery compared to the control samples only thawed once prior to be measured. Performance of the PRO-C17 ELISA was compared to the commercial type XVII ELISA kit (MyBioSource, San Diego, CA, USA) by measuring 8 serum samples from patients with non-small cell lung cancer (NSCLC) and 8 healthy individuals.

### Cell Culture of A-431 Cells

A-431 are epithelial cells derived from a patient with epidermoid carcinoma that constitutively express and cleave type XVII collagen. A-431 cell lines were purchased from ATCC (Rockville, MD, USA). The cells were cultured in two different conditions: keratinocyte SEM (KSFM; Gibco, United Kingdom) supplemented with human recombinant epidermal growth factor (rEGF), bovine pituitary extract (BPE) and 0.4 mM CaCl₂), and Dulbecco's Modified Eagle's Medium (DMEM, Gibco, United Kingdom) supplemented with 10% of fetal bovine serum (FBS; Gibco, United Kingdom).

### Western Blot of A-431 Cells

Supernatant and cell lysate from A-431 cells were run on a NuPAGE 4-12% Bis-Tris gel (Invitrogen, Carlsbad, CA, USA) under reducing conditions using a NuPAGE MES SDS running buffer (Invitrogen). The proteins from the polyacrylamide gel were transferred to an iBlot nitrocellulose membrane (Life technologies, Bengaluru, India) using an iBlot Dry blotting system (Life Technologies, Carlsbad, CA, USA). Next, the membrane was blocked for 60 min with 5% skim milk (Sigma-Aldrich, St. Louis, MO, USA) in TBST (Tris-buffered saline (TBS) with 0.1%, Tween-20). After that the membrane was incubated overnight with 0.001 mg/mL of the type XVII monoclonal antibody at 4° C. The day after the membrane was washed with TBST three times for 10 min in incubated right after with the secondary antibody (HRP-labelled) (1:5000) for 60 min. The membrane was washed with TBST three times for 10 min and incubated with SuperSignal west femto maximum sensitivity substrate (Thermo Fisher Scientific, Waltham, MA, USA) for 1-5 min and the protein bands were visualized on a C-DiGit Blot Scanner (LI-COR Biosciences, Lincoln, NE, USA). After development, the membrane was washed with TBST for 15 min. Stripping of the was performed by incubating the membrane with 15 mL of Restore Western Blot Stripping buffer (Thermo Fisher Scientific, Waltham, MA, USA) for 15 min. The membrane was then washed three times for 10 min with TBST to remove stripping buffer and re-blocked for 1 h using 30 mL of 5% skim milk in TBST. Thereafter the membrane was incubated with 1:1000 of commercial type XVII collagen antibody (MyBioSource, San Diego, CA, USA) and proceeded with the steps described before.

### Patient Samples

Cohort 1 consisted of 214 cancer patients and 23 healthy controls. It included 19 samples of bladder, breast and prostate cancer, 20 samples of colorectal, head and neck, kidney, melanoma, ovarian, pancreatic and stomach cancer, 17 samples of lung cancer and 23 age matched healthy controls. Cancer serum samples were purchased from Proteogenex (Los Angeles, CA, USA) and the healthy controls were obtained from BioIVT (Westbury, NY, USA). A summary of the cohort 1 characteristics can be found in table 1.

TABLE 1

| Characteristic | Cancer, N = 214 | Healthy, N = 23 |
|---|---|---|
| Diagnosis, n (%) | | |
| Bladder Cancer | 19 (8.9) | 0 (0) |
| Breast Cancer | 19 (8.9) | 0 (0) |
| Colorectal Cancer | 20 (9.3) | 0 (0) |
| Head & Neck Cancer | 20 (9.3) | 0 (0) |
| Kidney Cancer | 20 (9.3) | 0 (0) |
| Lung Cancer | 17 (7.9) | 0 (0) |
| Melanoma | 20 (9.3) | 0 (0) |
| Ovarian Cancer | 20 (9.3) | 0 (0) |
| Pancreatic Cancer | 20 (9.3) | 0 (0) |
| Prostate Cancer | 19 (8.9) | 0 (0) |
| Stomach Cancer | 20 (9.3) | 0 (0) |
| Healthy | 0 (0) | 23 (100) |
| Stages, n (%) | | |
| I | 7 (3.3) | 0 (0) |
| II | 45 (21) | 0 (0) |
| III | 89 (42) | 0 (0) |
| IV | 73 (34) | 0 (0) |
| N | 0 (0) | 23 (100) |
| Age, Mean (SD) | 59 (11) | 57 (6) |
| Sex, n (%) | | |
| Male | 115 (54) | 16 (70) |
| Female | 99 (46) | 7 (30) |

Cohort 2 comprised pre-treatment serum samples from 212 patients with metastatic colorectal cancer part of the study CREBB “ColoRectal cancer—Evaluation of Biomarkers in Bevacizumab treatment” from 2011 to 2016 from four Swedish and three Danish hospitals. Serum samples were collected at baseline before applying palliative treatment with chemotherapy combined with bevacizumab. The study was performed according to the recommendations of the Danish Regional Committee on Health Research Ethics. The CREBB protocol was approved by the Danish Regional Committee on Health Research Ethics (Approval ID: H-3-2010-121) and the Data Protection Agency (Approval ID: 2007-58-0015/HEH. 750.24-44). The study was conducted in accordance with the principles of the Declaration of Helsinki and all patients provided written informed consent prior to enrolment. Clinical data included: Age, Sex, total number of drugs, location, number of metastatic sites at study inclusion, performance status, synchronous metastatic disease and information about primary tumor resection. A summary of the cohort 2 characteristics can be found in table 2.

TABLE 2

| Characteristic | Healthy, N = 28¹ | Cancer, N = 212¹ | |
|---|---|---|---|
| Sex | | | |
| Female | 15 (54%) | 85 | (40%) |
| Male | 13 (46%) | 127 | (60%) |
| Age | | | |
| Mean (SD) | 54 (8) | 66 | (10) |
| Median (IQR) | 52 (49, 57) | 67 | (60, 73) |
| Range | 32, 72 | 31, 99 | |
| Location | | | |
| Right colon | 0 (0) | 69 | (33%) |
| Left colon and rectum | 0 (0) | 136 | (64%) |
| Appendix, small intestine | 0 (0) | 7 | (3.3%) |
| Unknown | 28 | 0 | |
| Sync | | | |
| No | 0 (0) | 64 | (30%) |
| Yes | 0 (0) | 148 | (70%) |
| Unknown | 28 | 0 | |
| OP | | | |
| Primary resected at baseline | 0 (0) | 101 | (48%) |
| Primary in situ at baseline | 0 (0) | 111 | (52%) |
| Unknown | 28 | 0 | |
| PS | | | |
| 0 | 0 (0) | 134 | (65%) |
| 1 | 0 (0) | 63 | (31%) |
| 2 | 0 (0) | 7 | (3.4%) |
| 3 | 0 (0) | 2 | (1.0%) |
| Unknown | 28 | 6 | |
| Mets | | | |
| 0 | 0 (0) | 3 | (1.4%) |
| 1 | 0 (0) | 79 | (37%) |
| 2 | 0 (0) | 95 | (45%) |
| 3 | 0 (0) | 27 | (13%) |
| 4 | 0 (0) | 8 | (3.8%) |
| Unknown | 28 | 0 | |
| Line | | | |
| 0 | 0 (0) | 1 | (0.5%) |
| 1 | 0 (0) | 127 | (60%) |
| 2 | 0 (0) | 69 | (33%) |
| 3 | 0 (0) | 12 | (5.7%) |
| 4 | 0 (0) | 2 | (0.9%) |
| 9 | 0 (0) | 1 | (0.5%) |
| Unknown | 28 | 0 | |
| Total drugs in regimen | | | |
| 2 | 0 (0) | 58 | (27%) |
| 3 | 0 (0) | 151 | (71%) |
| 4 | 0 (0) | 3 | (1.4%) |
| Unknown | 28 | 0 | |
| Baseline CEA | | | |
| Mean (SD) | 0 (0) | 413 | (1,775) |
| Median (IQR) | 0 (0) | 26 | (7, 93) |
| Range | 0 (0) | 1, 14,900 | |
| Unknown | 28 | 67 | |
| Baseline Leuko | | | |
| Mean (SD) | 0 (0) | 7.44 | (2.74) |
| Median (IQR) | 0 (0) | 7.00 | (5.50, 8.80) |
| Range | 0 (0) | 2.10, 16.70 | |
| Unknown | 28 | 5 | |
| Baseline Trombo | | | |
| Mean (SD) | 0 (0) | 283 | (113) |
| Median (IQR) | 0 (0) | 270 | (207, 332) |
| Range | 0 (0) | 3, 831 | |
| Unknown | 28 | 5 | |
| Baseline Neutro | | | |
| Mean (SD) | 0 (0) | 5.01 | (2.71) |
| Median (IQR) | 0 (0) | 4.37 | (3.26, 6.00) |
| Range | 0 (0) | 0.74, 15.30 | |
| Unknown | 28 | 55 | |
| Baseline LDH | | | |
| Mean (SD) | 0 (0) | 275 | (213) |
| Median (IQR) | 0 (0) | 208 | (176, 276) |
| Range | 0 (0) | 85, 1,659 | |
| Unknown | 28 | 24 | |
| Baseline PRO-C3 | | | |
| Mean (SD) | 0 (0) | 24 | (27) |
| Median (IQR) | 0 (0) | 13 | (11, 23) |
| Range | 0 (0) | 5, 237 | |
| Unknown | 28 | 0 | |
| Baseline PRO-C17 | | | |
| Mean (SD) | 1.59 (0.51) | 2.26 | (0.82) |
| Median (IQR) | 1.53 (1.30, 1.86) | 2.25 | (1.80, 2.66) |

| | | |
|---|---|---|
| Range | 0.86, 3.17 | 0.86, 5.64 |
| Unknown | 0 | 1 |

### Statistics

In cohort 1 comparisons of PRO-C17 levels in the different cancer patients compared to the healthy controls was determined by performing a one-way ANOVA followed by multiple comparisons using T-test. The diagnostic accuracy of the assay was assessed with the area under the receiver operating characteristics (AUROC) curve. In cohort 2, PRO-C17 levels in metastatic colorectal patients were compared to PRO-C17 levels in healthy controls with t-test. The overall survival (OS) in patients with low PRO-C17 levels (tertile 1+tertile 2) was compared to patients with high PRO-C17 levels (tertile 3) with Kaplan-Meier curves and log-rank test was applied to determine differences between the curves. Multivariate Cox regression analysis stratified by line of treatment was performed to evaluate the independent predictive value of PRO-C17 when adjusted for age, sex, total number of drugs, location, synchronous metastatic disease, performance status, primary tumor resection, number of metastatic sites and PRO-C3 levels at baseline. Significance was considered with p-values <0.05 as it follows: * p<0.05; ** p<0.01; ** * p<0.001; p<0.0001. Statistical analyses were made with GraphPad Prism (version 9.5.0 for Windows, GraphPad Software, San Diego, California USA, www.graphpad.com) and R version 4.2.2 (R Core Team (2022), R Foundation for Statistical Computing, Vienna, Austria, https://www.R-project.org).

### Results

### PRO-C17 ELISA Development

Optimization of the assay protocol included best time (20 hours) and antibody incubation temperature (4° C.) as well as choice of assay buffer (50 mM PBS, 1% BSA (w/v), 0.018% bronidox (v/v), 0.1% Tween-20 (w/v), 8 g/L NaCl, pH 7.4) and concentration of the other kit components. The final assay parameters were selected according to which provided the highest sensitivity in human serum while adhering to the technical specifications listed below.

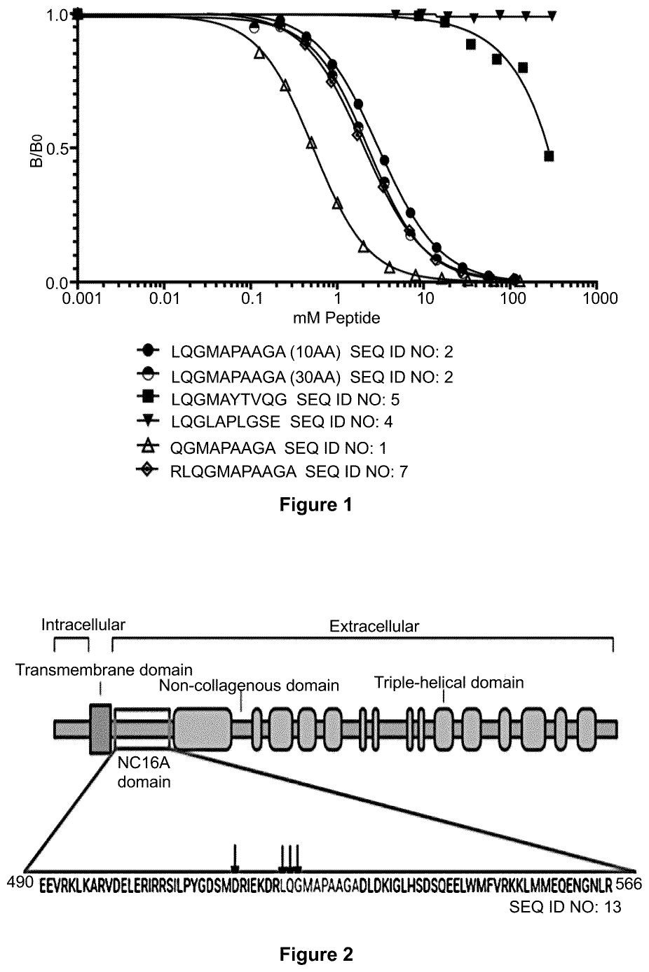

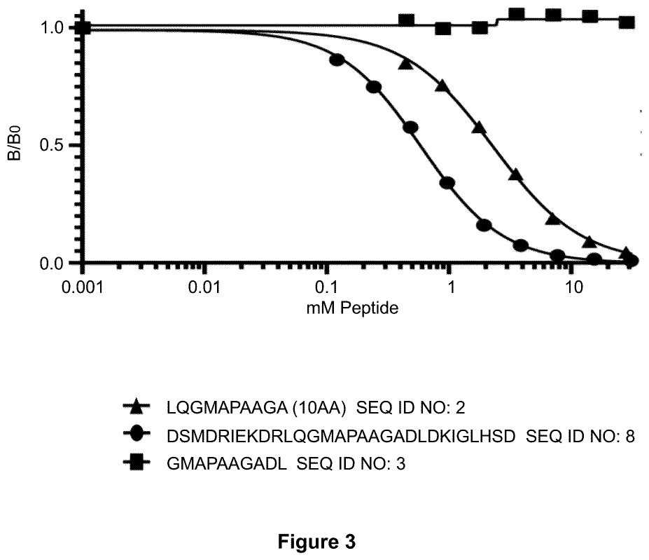

The specificity of the assay was assessed by the ability of different peptides to compete for binding to the monoclonal antibody in a competitive ELISA format. The group of peptides included the 30 amino acid standard peptide, corresponding to an internal epitope of the NC16A domain of type XVII collagen (LQGMAPAAGADLDKIGLHSDSQEELWMFVRK), an elongated peptide (RLQGMAPAAGA), a truncated peptide (QGMAPAAGA) and two deselection peptides (LQGMAYTVQG and LQGLAPLGSE) (FIG. 1). Even though no cross reactivity was observed towards the two deselection peptides the both the elongated and the truncated peptide were recognized by the antibody (FIG. 1). Epitope mapping was performed to test antibody affinity towards neighbouring epitopes corresponding to peptides originated from the different physiological cleavage sites in the NC16A domain (FIG. 2), which included an further elongated version of the selected peptide (DSMDRIEKDRLQGMAPAAGADLDKIGLHSD) and a further truncated one (GMAPAAGADL). The antibody recognized DSMDRIEKDRLQGMAPAAGADLDKIGLHSD whereas no cross reactivity was observed towards GMAPAAGADL, suggesting that the antibody is not neo-epitope specific but sequence specific (FIG. 3).

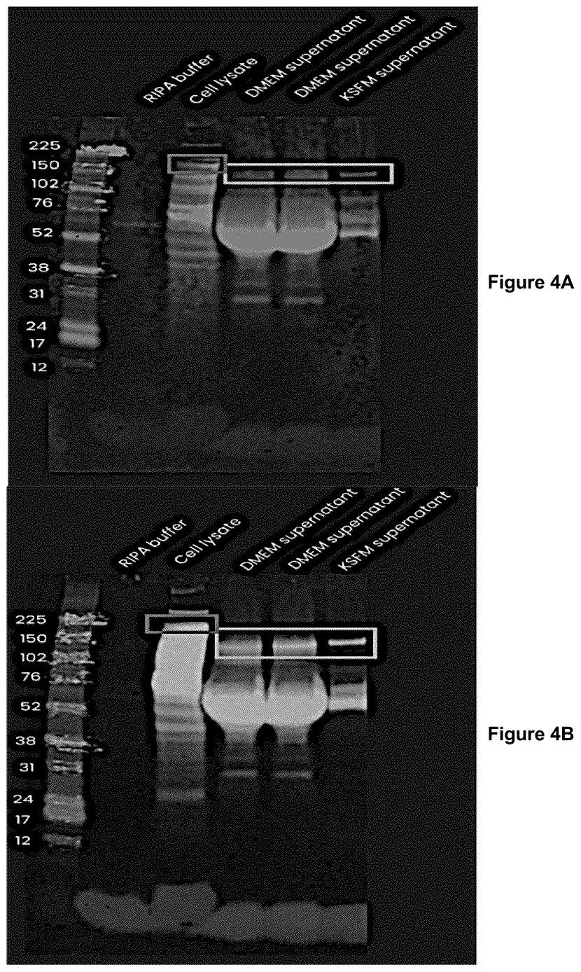

Western blot analysis showed that type XVII collagen ectodomain (120 kDa) was present in the A-431 supernatant, whereas the full fragment of type XVII (180 kDa) was only detected in the cell lysate (FIG. 4). Linearity of dilution was accepted from 1:2 to 1:16. Matrix-in-matrix spiking test for accuracy testing showed a recovery of 99.6%. Interference from common endogenous analytes was observed, but within the accepted recovery rates (80-120%) for all samples. Intra- and inter-assay variation were 4.2% and 9.3% respectively. Analyte stability was evaluated for up to 48 hours at 4 or 20° C., with a mean recovery of 102.38. Stability after four freeze-thaw cycles had a 95.2% recovery rate.

The different technical validation steps are summarized in table 3.

TABLE 3

| Assay parameter | Result | |
|---|---|---|
| ELISA format | Competitive ELISA with TMB | |
| Intended matrix (MRD) | Human serum (1 + 1) | |
| Incubation buffer | 50 mM PBS-BTB, 8 g/L NaCl | |
| Measurement range: LLOQ-ULOQ | 0.43-15 (ng/mL) uncorrected | |
| | 0.86-30 (ng/mL) corrected | |
| LLOB | 0.33 | ng/mL |
| Mean slope | 1.1 | |
| Mean IC25 | 0.8 | ng/mL |
| Mean IC50 | 2.1 | ng/mL |
| Mean IC75 | 5.5 | ng/mL |

| | |
|---|---|
| Intra-assay CV % | 4% |
| Inter assay CV % | 9.3% |
| Dilution recovery (1 + 1 from MRD) | 103.7% |
| Accepted maximum freeze-thaw | 99% |
| Interference lipid, low/high | 102%/93% |
| Interference hemoglobin, low/high | 96%/93% |
| Interference biotin, low/high | 101%/107% |
| Analyte stability (48 h 4° C./ | 103%/107% |
| 48 h 20° C.) | |

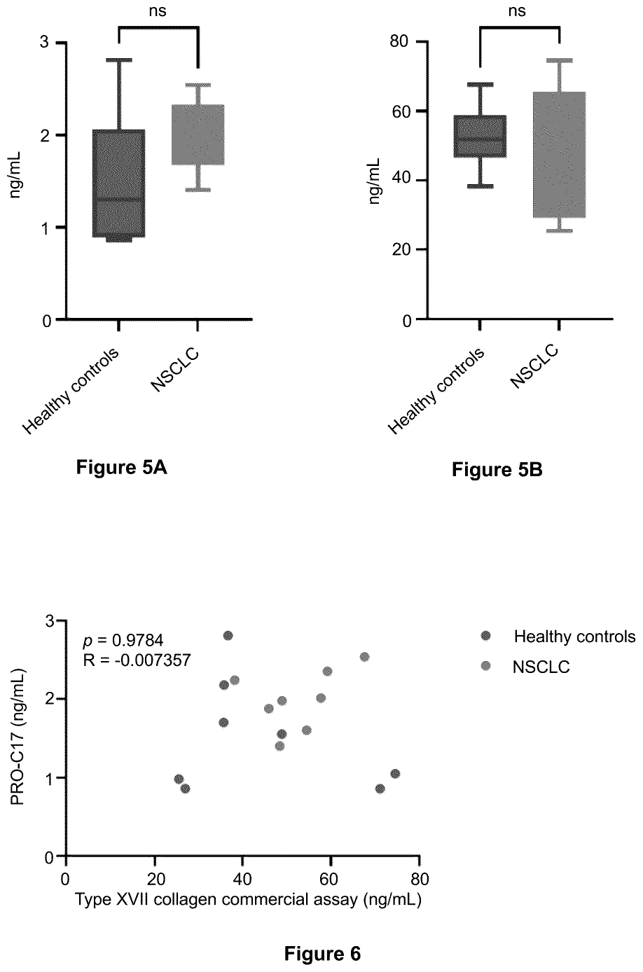

When measuring serum samples from patients with non-small cell lung cancer (NSCLC) even though not significant (p >0.05), the PRO-C17 assay showed increased levels of type XVII 5 collagen compared to healthy individuals whereas the commercial kit showed the opposite trend (FIG. 5). Moreover, there was no correlation of type XVII collagen levels in the two assays (r=−0.007357, p=0.9784) suggesting that the two assays target different epitopes of type XVII (FIG. 6).

PRO-C17 in Patients with Multiple Cancers

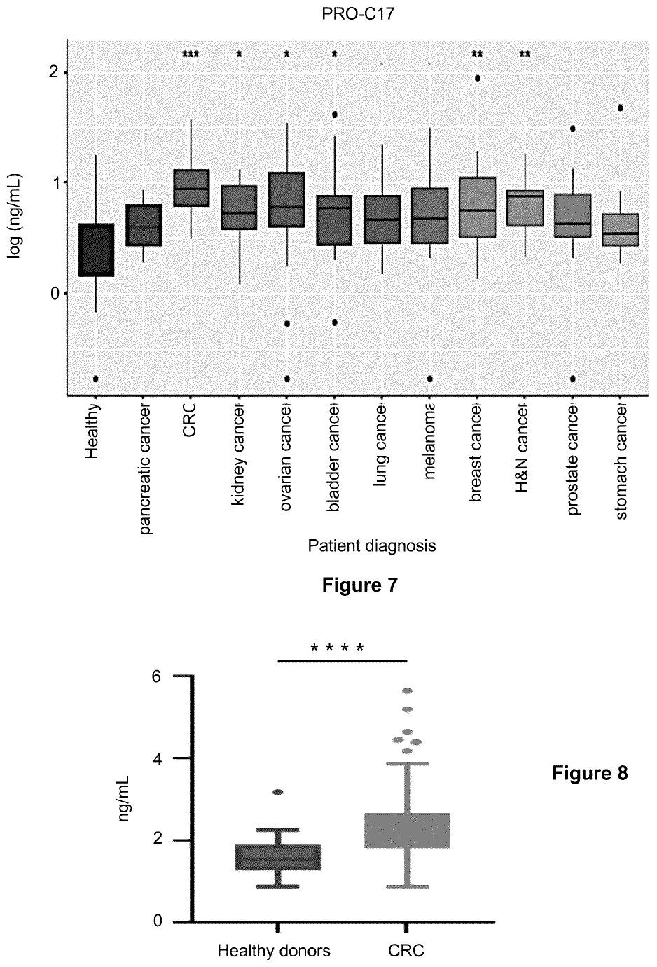

In cohort 1, PRO-C17 levels were significantly elevated compared to healthy controls (p<0.05) in serum from patients with colorectal cancer, kidney cancer, ovarian cancer, bladder cancer, breast cancer, and head and neck cancer. They were also visually (but not statistically significantly) elevated in serum from patients with pancreatic cancer, lung cancer, melanoma, prostate cancer, and stomach cancer (FIG. 7).

The AUROC values for the different cancer types are shown in table 4.

TABLE 4

| Cancer type | AUROC |
|---|---|
| Bladder cancer | 0.742 |
| Breast cancer | 0.777 |
| Colorectal cancer | 0.904 |
| Head & neck cancer | 0.828 |
| Kidney cancer | 0.789 |
| Lung cancer | 0.739 |
| Melanoma | 0.737 |
| Ovarian cancer | 0.759 |
| Pancreatic cancer | 0.722 |
| Prostate cancer | 0.727 |
| Stomach cancer | 0.690 |

PRO-C17 was especially good at discriminating between colorectal patients and healthy controls with an AUROC of 0.904 (Table 4). These results suggest that circulating levels of PRO-C17 are elevated in several cancer types and that PRO-C17 may be particularly relevant for colorectal cancer.

PRO-C17 in Serum Patients with Metastatic Colorectal Cancer Predicts Poor Outcome

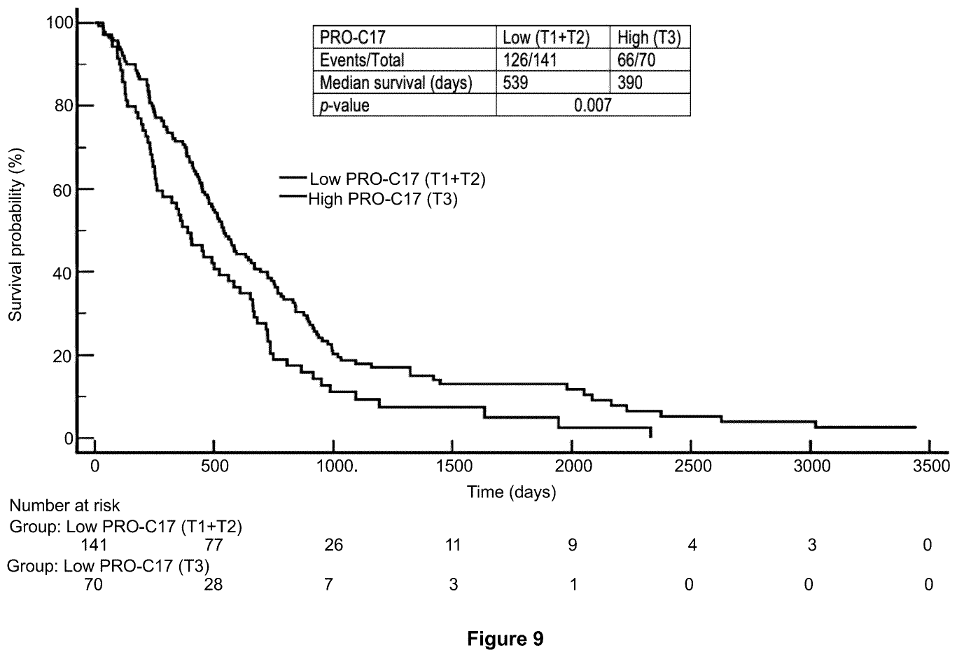

In cohort 2, it was found that PRO-C17 levels are increased in serum from patients with metastatic colorectal cancer compared to healthy subjects (p<0.0001) (FIG. 8). To investigate the prognostic value of PRO-C17, the patients in cohort 2 were separated into two groups according to biomarker levels: “low” (T1+T2) and “high” (T3). The Kaplan-Meier survival analysis showed that patients with low PRO-C17 levels had a median overall survival (OS) of 539 days compared to the 390 days in patients with high PRO-C17 levels (log-rank p=0.007) (FIG. 9). The ability of PRO-C17 to predict OS was then investigated with the multivariate Cox proportional-hazards model stratified by line of treatment.

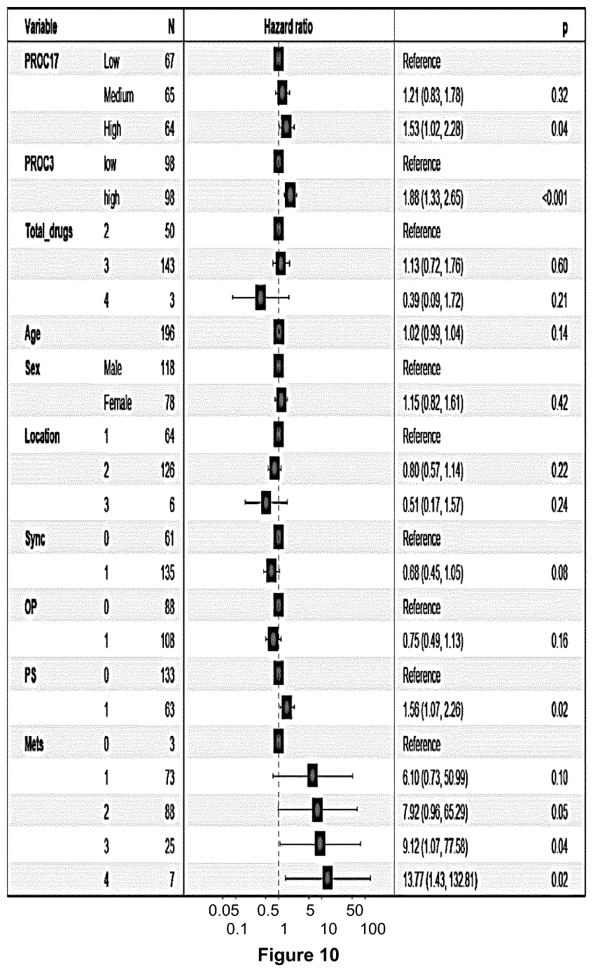

When PRO-C17 was adjusted for the covariates age, sex, total number of drugs, synchronous metastatic disease, PRO-C3 levels at baseline, location of the tumor, operation status, performance status and number of metastatic sites, high PRO-C17 levels were predictive of poor OS and increased risk of dying of 53% (HR=1.53, 95% CI=1.02, 2.28, p=0.04) (FIG. 10).

### CONCLUSION

The inventors have successfully developed, optimized and validated an ELISA to quantify type XVII collagen fragments in blood (the PRO-C17 assay). The PRO-C17 assay is a technically robust, precise and sensitive assay. The inventors have further found that levels of PRO-C17 were increased in patients with cancer and in particular colorectal cancer compared to healthy controls. Moreover, in metastatic colorectal patients treated with bevacizumab in combination with chemotherapy high levels of the biomarker was predictive of poor overall survival and increased risk of dying.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

[1] World Health Organization, “Cancer,” https://www.who.int/en/news-room/fact-sheets/detail/cancer, Feb. 3, 2022.
[2] H. Denys et al., “The Extracellular Matrix Regulates Cancer Progression and Therapy Response: Implications for Prognosis and Treatment,” Curr Pharm Des, vol. 15, no. 12, pp. 1373-1384 April 2009, doi: 10.2174/138161209787846711.
[3] Karsdal M., Biochemistry of collagens: structure, function and biomarkers, 2nd ed. Academic Press, 2019.
[4] P. Lu, V. M. Weaver, and Z. Werb, “The extracellular matrix: a dynamic niche in cancer progression.,” J Cell Biol, vol. 196, no. 4, pp. 395-406, February 2012, doi: 10.1083/jcb.201102147.
[5] M. D. Shoulders and R. T. Raines, “Collagen Structure and Stability,” Annu Rev Biochem, vol. 78, no. 1, pp. 929-958, June 2009, doi: 10.1146/annurev.biochem.77.032207.120833.
[6] S. Ricard-Blum, “The Collagen Family,” Cold Spring Harb Perspect Biol, vol. 3, no. 1, pp. a004978-a004978, January 2011, doi: 10.1101/cshperspect.a004978.
[7] R. Heljasvaara, M. Aikio, H. Ruotsalainen, and T. Pihlajaniemi, “Collagen XVIII in tissue homeostasis and dysregulation-Lessons learned from model organisms and human patients,” Matrix Biology, vol. 57-58, pp. 55-75, January 2017, doi: 10.1016/j.matbio.2016.10.002.
[8] A. Heikkinen, H. Tu, and T. Pihlajaniemi, “Collagen XIII: A type II transmembrane protein with relevance to musculoskeletal tissues, microvessels and inflammation,” Int J Biochem Cell Biol, vol. 44, no. 5, pp. 714-717, May 2012, doi: 10.1016/j.biocel.2012.01.024.
[9] J. E. Murphy-Ullrich and E. H. Sage, “Revisiting the matricellular concept,” Matrix Biology, vol. 37, pp. 1-14, July 2014, doi: 10.1016/j.matbio.2014.07.005.
[10] G. J. Giudice, D. J. Emery, and L. A. Diaz, “Cloning and Primary Structural Analysis of the Bullous Pemphigoid Autoantigen BP180,” Journal of Investigative Dermatology, vol. 99, no. 3, pp. 243-250, September 1992, doi: 10.1111/1523-1747.ep12616580.
[11] A. Snellman, H. Tu, T. Väisänen, A.-P. Kvist, P. Huhtala, and T. Pihlajaniemi, “A short sequence in the N-terminal region is required for the trimerization of type XIII collagen and is conserved in other collagenous transmembrane proteins,” EMBO J, vol. 19, no. 19, pp. 5051-559 October 2000, doi: 10.1093/emboj/19.19.5051.
[12] T. Hashimoto, “CLAC: a novel Alzheimer amyloid plaque component derived from a transmembrane precursor, CLAC-P/collagen type XXV,” EMBO J, vol. 21, no. 7, pp. 1524-1534 April 2002, doi: 10.1093/emboj/21.7.1524.
[13] J. Banyard, L. Bao, and B. R. Zetter, “Type XXIII Collagen, a New Transmembrane Collagen Identified in Metastatic Tumor Cells,” Journal of Biological Chemistry, vol. 278, no. 23, pp. 20989-20994, June 2003, doi: 10.1074/jbc.M210616200.
[14] T. Wakabayashi, “Transmembrane Collagens in Neuromuscular Development and Disorders,” Front Mol Neurosci, vol. 13, January 2021, doi: 10.3389/fnmol.2020.635375.
[15] J. A. McGrath et al., “Mutations in the 180-kD bullous pemphigoid antigen (BPAG2), a hemidesmosomal transmembrane collagen (COL17A1), in generalized atrophic benign epidermolysis bullosa,” Nat Genet, vol. 11, no. 1, pp. 83-86, September 1995, doi: 10.1038/ng0995-83.
[16] E. Schmidt and D. Zillikens, “Pemphigoid diseases,” The Lancet, vol. 381, no. 9863, pp. 320-332, January 2013, doi: 10.1016/S0140-6736 (12) 61140-4.
[17] W. Nishie, “Update on the pathogenesis of bullous pemphigoid: An autoantibody-mediated blistering disease targeting collagen XVII,” J Dermatol Sci, vol. 73, no. 3, pp. 179-186, March 2014, doi: 10.1016/j.jdermsci.2013.12.001.
[18] K. Tasanen, L. Tunggal, G. Chometon, L. Bruckner-Tuderman, and M. Aumailley, “Keratinocytes from Patients Lacking Collagen XVII Display a Migratory Phenotype,” Am J Pathol, vol. 164, no. 6, pp. 2027-238 June 2004, doi: 10.1016/S0002-9440 (10) 63762-5.
[19] J. M. Moilanen et al., “Collagen XVII expression correlates with the invasion and metastasis of colorectal cancer,” Hum Pathol, vol. 46, no. 3, pp. 434-442, March 2015, doi: 10.1016/j.humpath.2014.11.020.
[20] S. Laval et al., “Dual roles of hemidesmosomal proteins in the pancreatic epithelium: the phosphoinositide 3-kinase decides,” Oncogene, vol. 33, no. 15, pp. 1934-1944 April 2014, doi: 10.1038/one.2013.146.
[21] P. U. Thangavelu, T. Krenacs, E. Dray, and P. H. G. Duijf, “In epithelial cancers, aberrant COL17A1 promoter methylation predicts its misexpression and increased invasion,” Clin Epigenetics, vol. 8, no. 1, p. 120, December 2016, doi: 10.1186/s13148-016-0290-6.
[22] M. Parikka, T. Kainulainen, K. Tasanen, A. Väänänen, L. Bruckner-Tuderman, and T. Salo, “Alterations of Collagen XVII Expression During Transformation of Oral Epithelium to Dysplasia and Carcinoma,” Journal of Histochemistry & Cytochemistry, vol. 51, no. 7, pp. 921-929, July 2003, doi: 10.1177/002215540305100707.
[23] Y. Ohta et al., “Cell-matrix interface regulates dormancy in human colon cancer stem cells,” Nature, vol. 608, no. 7924, pp. 784-794, August 2022, doi: 10.1038/s41586-022-05043-y.
[24] Nishie W, Lamer S, Schlosser A, Licarete E, Franzke CW, Hofmann SC, Jackow J, Sitaru C, Bruckner-Tuderman L. “Ectodomain shedding generates Neoepitopes on collagen XVII, the major autoantigen for bullous pemphigoid.” J Immunol. 2010 Oct. 15; 185(8):4938-47. doi: 10.4049/jimmunol.1001524.

1: A method of immunoassay for detecting and/or monitoring a cancer in a patient and/or assessing the severity of a cancer in a patient, the method comprising;
i) contacting a patient sample with a monoclonal antibody that specifically binds to the amino acid sequence QGMAPAAGA (SEQ ID NO: 1);
ii) detecting and determining the amount of binding between the monoclonal antibody and peptides in the sample; and
iii) correlating the amount of binding with values associated with normal healthy subjects and/or values associated with known disease severity and/or values obtained from the patient at a previous time point and/or with a predetermined cut-off value.

2: The method of claim 1, wherein the monoclonal antibody specifically binds to the amino acid sequence LQGMAPAAGA (SEQ ID NO: 2).

3: The method of claim 1, wherein the monoclonal antibody does not specifically bind to the amino acid sequence GMAPAAGADL (SEQ ID NO: 3).

4: The method of claim 1, wherein the monoclonal antibody does not specifically bind to the amino acid sequence LQGLAPLGSE (SEQ ID NO: 4).

5: The method of claim 1, wherein the monoclonal antibody does not specifically bind to the amino acid sequence LQGMAYTVQG (SEQ ID NO: 5).

6: The method of claim 1, wherein the monoclonal antibody is raised against a synthetic peptide comprising the amino acid sequence LQGMAPAAGA (SEQ ID NO: 2).

7: The method of claim 1, wherein the cancer is pancreatic cancer, colorectal cancer, kidney cancer, ovarian cancer, bladder cancer, lung cancer, melanoma, breast cancer, head and neck cancer, prostate cancer or stomach cancer.

8: The method of claim 7, wherein the cancer is colorectal cancer.

9: The method of claim 1, wherein the method is a method of immunoassay for assessing the severity of a cancer in a patient.

10: The method of claim 1, wherein the patient sample is selected from blood, serum or plasma.

11: The method of claim 9, wherein the immunoassay is a competition assay or a sandwich assay.

12: The method of claim 9, wherein the immunoassay is a radio-immunoassay or an enzyme-linked immunosorbent assay.
